(19) United States
(12) Patent Application Publication
(10) Pub. No.: US 2001/0034489 A1
(43) Pub. Date: Oct. 25, 2001

(54) Computerized animal EKG analysis system (2) [AEMPI (2)]

(76) Inventor: Genquan Feng, New York, NY (US)

Correspondence Address: Prof. Genquan Feng, P.O. Box 1796, New York, NY, 10185-0016, US

(21) Appl. No.: 09/802,007

(22) Filed: Mar. 9, 2001

Related U.S. Application Data
(63) Continuation-in-part of application No. 09/130,047, filed on Aug. 6, 1998, now abandoned.

(51) Int. Cl. A61B 5/04

(52) U.S. Cl. 600/515

(57) ABSTRACT

Animal EKG analysis is still done by veterinary doctors, it is not only difficult but also subjective. EMPI (EKG Multiphase Information Diagnosis) Technology has been successfully used for the human heart, it is reasonably think that use this new technology into animal EKG analysis shall be effective and makes the animal EKG analysis to be automatic and objective. This patent introduces the inventor's new invention for computerized analysis of animal EKG based on the computerized EKG wave-form analysis technology and EMPI technology.

### BACKGROUND OF THE INVENTION

1. Field of the Invention

This invention generally relates to a method of, and an arrangement for, determining a condition of a sample to be analyzed and, more particularly, to detect and differentiate the dysfunction of the animal heart.

2. Description of the Related Art

Detection of dysfunction of the animal heart not only useful in the veterinary field, but also for use in clinical or pre-clinical drug studies for screening of pharmaceutical performance of drugs, and also for other uses such as detection of some effect of environmental factors (pollution, radiation, etc.) to the animal heart, which is usually used for derivation or prediction of the effect to the human heart.

Computerized EKG (Electrocardiogram) system for interpretation of human EKG prevailed to be used in the medical field in last few decades. But until now, the interpretation of animal EKG data still mainly depend on visually analyzed by veterinary experts, it is not only waste time but also can't avoid the subjective effect of the interpreter. The same animal EKG sample can be got different interpretation by different veterinarians, which seriously affects the effectiveness of diagnosis and treatment of dysfunction of the animal heart and the efficacy of drug screening studies and environmental factors studies.

The prior art has proposed several approaches for computerized interpretation of human and animal EKG, such as U.S. Pat. No. 5,029,082, teaches a plurality of functions to describe of the heart dysfunction of the patient which are mathematically determined by a computerized system (regardless some substantial problems of that patent). U.S. Pat. No. 5,694,544 teaches by using of transforming the EKG signals to plural domains (multiphase) for early detection of heart dysfunction, etc.

The problem is all those prior arts are mainly for analysis of human EKG, but animal EKG is not so identical to human heart, the methods for analysis of human EKG signals, especially the indexes, usually are not sufficient for precisely detection of the dysfunction of animal heart. Therefore, invention of a set of method and arrangement for objective and sufficient precise to detect the dysfunction of animal heart is important for the drug screening, environmental study and veterinary field currently

### SUMMARY OF THE INVENTION

1. Objects of the Invention

It is a general object of this invention to advance the state of the diagnostic art for detecting dysfunction of the animal heart.

Another object of this invention is to detect dysfunction of the animal heart in an early stage in its progress.

Still another object of this invention is to non-invasively and accurately to diagnose dysfunction of the animal heart.

A further object of this invention is to differentially diagnose different type of dysfunction of the animal heart.

2. Feature of the Invention

In keeping with these objects and others, which will become apparent hereinafter, one feature of this invention relates. In its broadest aspect, to determining a condition of a sample by acquiring electrical analog signals from the sample. In one preferred embodiment, the signals are EKG signals obtained from a surface of the body of an animal by placement of a plurality of surface electrodes at various sites thereon.

The method and arrangement of this invention, however, are not intended to be limited to the determination of heart dysfunction from EKG signals. This invention can be extended to the analysis of any biological signals generated during the course of such medical examinations as an electromyogram (EMG), electrobasogram, eletroencephalogram (EEG), electrogastrogram, electrocystogram, electrocorticogram, eletrometrogram, electronystagmogram, electro-oculogram, electroretinogram, etc. In addition, the method and arrangement of this invention can be extended to the analysis of non-biological signals, e.g. physical signals or chemical signals, obtained during the course of measurement in a scismogram, electophoretogram, thermogram, etc.

This invention processes the analog signals, whether biological or not, and mathematically determines a plurality of functions descriptive of the sample being analyzed. Thus, in the case of EKG signals, the functions include, as described in detail below, the power spectrum characteristic, the coherence characteristic, the phase angle characteristic, the impulse response characteristic, the cross correlation characteristic and the amplitude histogram characteristic. Each of these functions carries a wealth of different information about the EKG signals, particularly when the functions are processed over an extended time period which, in; the preferred embodiment, is 15 cycles lasting 10 seconds per cycle. The extended time period is many orders of magnitude greater than the typical analysis of EKG signals which, at best, process a minor fraction of one heart cycle of heart function characteristic.

In accordance with this invention, a set of indices is established for each function. Each index has two states. The positive state indicates an abnormal condition for the sample. A negative state indicates a normal condition. The indices generally relate to the pattern or shape of the wave-form of each function characteristic. As described in detail below, the preset indices include the magnitude of peaks, the intervals between peaks, the curvature of the peaks, the number of bends, etc.

An integrated index pattern of the states of the indices derived from a plurality. If not all, of the functions is generated. This integrated pattern is then matched against a stored collection of index patterns whose condition (i.e. diagnosis) is known. The best match then determines the diagnosis for the animal being analyzed.

The collection of stored index patterns is based on storing the index patterns of a multitude, e.g. several hundreds of animals whose condition is known and whose condition was confirmed by medical examination and testing. Thus, animals having ischemia, for example, have index patterns which, when grouped together, have a distinctive pattern. Animals having a different heart dysfunction have differently distinctive index patterns. As previously mentioned, the index pattern of the animal being tested is compared in each group of known patterns to find the best match and, hence, the diagnosis.

In the case of a cardiac dysfunction animal, the collection of stored index patterns are advantageously grouped into the following several categories, ischemia, arrythmia, Q-T prolong, etc. This invention thus can match the cardiac dysfunction animal's measured pattern against the patterns of these categories to select the one that best describes the dysfunctional animal's cardiac problem.

Hence, rather than relying on data extracted over a limited time for a single function, this invention relies on data extracted over an extended time from a plurality of functions, some of which have not priory been used in the diagnosis of animal heart dysfunction.

The novel features which are considered as characteristic of the invention are set forth in particular in the appended claims. The invention itself, however, both as to its construction itself, however, both as to its construction and its method of operation. Together with additional objects and advantages thereof, best will be understood from the following description of specific embodiments.

### BRIEF DESCRIPTION OF THE DRAWINGS

FIG. 1 is a front view of an arrangement in accordance with the method of this invention.

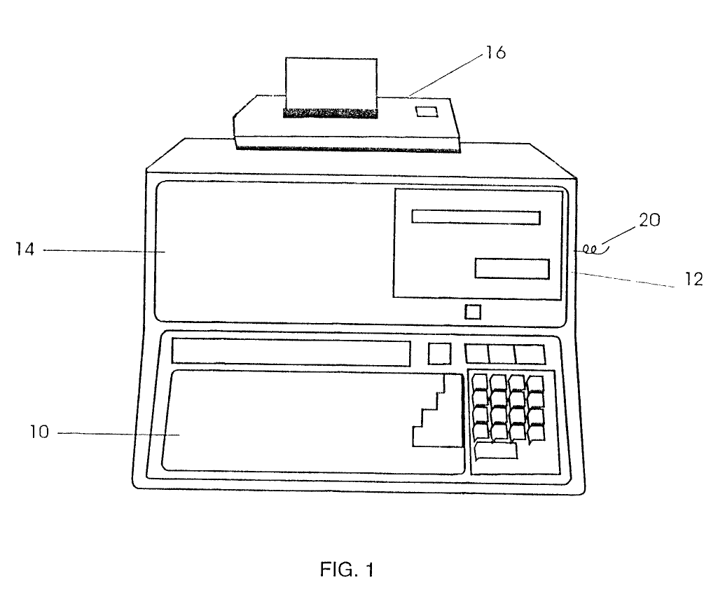

FIG. 2 is a block diagram of the arrangement of FIG. 1 connected to an animal.

FIG. 3 is an overall block diagram of the signal processor depicted in FIG. 2.

### DETAIL DESCRIPTION OF THE PREFERRED EMBODIMENTS

Referring now to the drawings, reference numeral 12 generally identifies an arrangement for diagnosing animal heart dysfunction in accordance with the method of this invention. Arrangement 12 includes a keyboard 10 for manual data entry and operational control, a monitor 14 for displaying and plotting data and a printer 16 for printing a written data record. As described below, electronic circuitry within the arrangement is employed to process animal EKG signals in order to obtain a diagnosis of a condition of an animal's heart.

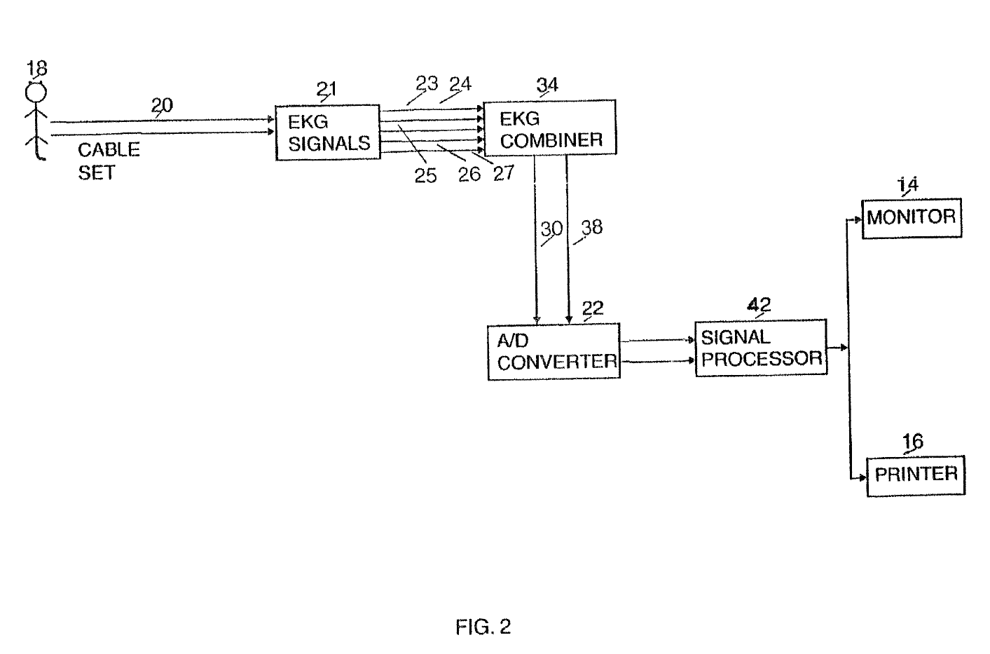

In the case of diagnosing heart dysfunction, the arrangement 12 is connected to the animal by a cable set 20 in a conventional EKG hook-up. The cable set 20 includes five wires each having a surface electrode positioned at various fixed sites on the animal's body. As depicted in FIG. 2. And electrode 23 connected to a conventional EKG “white” wire in placed over the area of the animal's body overlying the left ventricle. An electrode 24 connected to a conventional EKG “Yellow” wire is placed over the left hand. An electrode 25 connected to a conventional EKG “red” wire is placed over the right hand. An electrode 26 connected to a conventional EKG “green” wire is placed over the left leg. An electrode 27 connected to a conventional EKG “black” wire is placed over the right leg.

These electrodes 23-27 generate time-dependent electrical analog signals, as represented by block 21 in FIG. 2. These signals are fed into and combined in a novel manner in combiner network 34. The combiner network 34 combines the fore-mentioned five EKG signals into a pair of output signals at output 30, 38. The output signal at output 30 is conventionally designated hereinafter as “lead V5” and is indicative of the activity of the left ventricle. The output signal at output 38 is conventionally designated hereinafter as “lead II” and is indicative of the activity of a broad area of the heart. The various input signals from electrodes 23-27 are amplified in differential amplifiers within the EKG combiner 34.

At best shown in FIG. 2, the output analog EKG signals at outputs 30, 38 are sampled and digitized in an analog-to-digital converter 22. The digital signals are processed by a programmed microcomputer or signal processor 42. The results of the signal processing, as described below. Are displayed on monitor 14 or printed by printer 16.

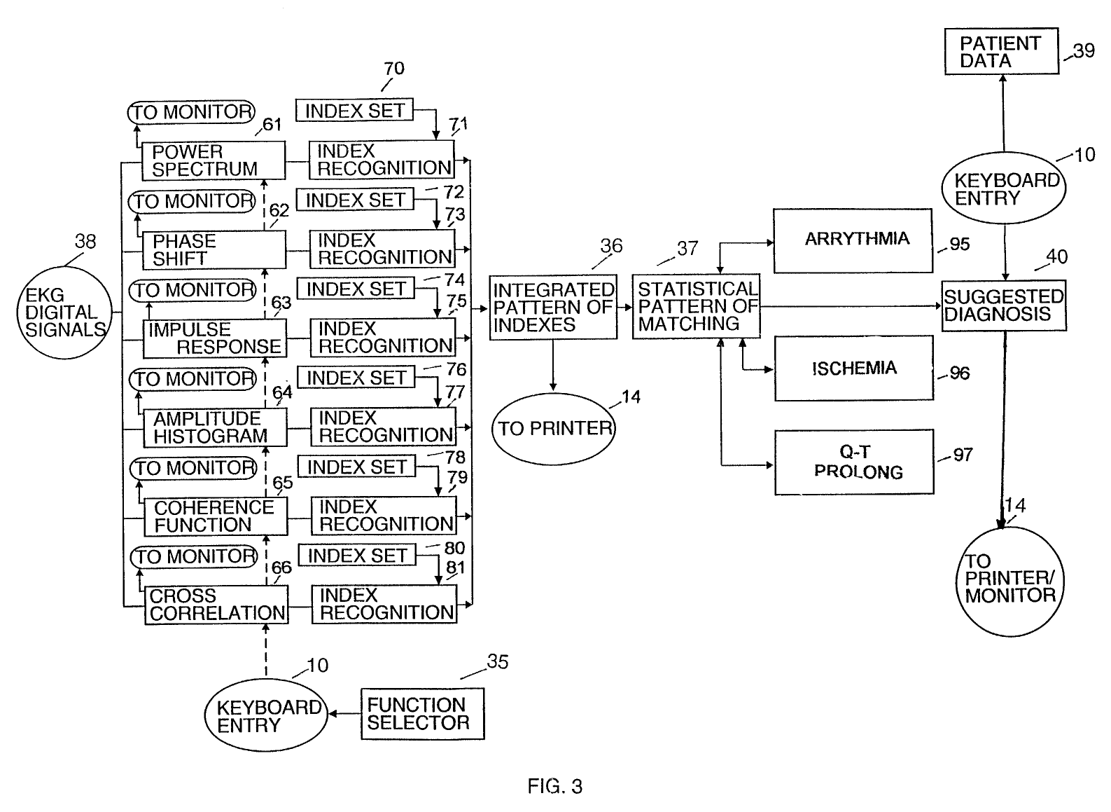

An overview of the signal processing is depicted in FIG. 3. The digital EKG signals from output 36, 38 are fed to function blocks 61-66 wherein the power spectrum, phase angle, impulse response, amplitude histogram, coherence and cross-correlation are respectively mathematically determined. In a preferred embodiment, all of these functions are determined and used in making the diagnosis. However, it is still useful, if a good “wave-form identification program” has been used for differentiate the indexes of the regular EKG signals, even none of the functions mentioned above are determined. But if use these functions (at least one), it shall be better for increase the accuracy of the diagnosis.

In FIG. 3, after the functions 61-66 have been calculated, they may be sequentially displayed on the monitor for evaluation by a technician, or, preferably, the function wave-forms, which are stored in a random access memory and subjected to battery of tests in which the presence or absence of various indices are recognized. These indices, all relate to the overall shape of the various function wave-forms and are established in advance. Each function has its own individual pre-set indices, as represented by blocks 70, 72, 74, 76, 78, 80 in FIG. 3. Each of these indices has two states. A positive state indicates an abnormal condition. A negative state indicates a normal condition. The recognition of the indices occurs in a pattern recognition program as represented by blocks 71, 73, 75, 77, 79, 81.

Once the state of each index has been recognized, an integrated pattern 36 is generated. The integrated pattern contains the states of the indices from at least two, if not all the above functions. The integrated pattern 36 can be printed out by the printer.

Once the integrated pattern 36 is generated, it is fed into a statistical pattern matching program 37 to which a massive data bank 95, 96, 97 is connected. The data bank includes a multitude of index patterns taken from thousands of animals whose heart condition is known, usually by direct medical examination. The index patterns of different dysfunction have different index sequences. Once the best match between the measured integrated pattern 36 and one of the stored patterns 95, 96, 97 is obtained, a diagnosis 40 is made.

Advantageously, in the case of cardiac analysis, the data bank is separated into several distinctive categories, namely arrythmia 95, ischemia 96, Q-T prolong 97. This invention can thus distinguish between these different types of animal heart dysfunction.

I claim:

1. A method of diagnosing a cardiac condition of an animal comprising the steps of
(a) acquiring electrocardiographic signals from the animal,
(b) mathematically determining the function descriptive of the animal heart from the electrocardiographic signals;
(c) establishing a set of indices for each function each indicative of the cardiac condition of the animal;
(d) generating an integrated pattern of the states of the indices from a plurality of the functions;
(e) recognizing the state of each index for each function;
(f) storing a collection of index patterns, each containing a multitude of patterns of the states of indices for a multitude of animals whose cardiac condition is known; and
(g) matching the generated integrated pattern against the stored collection of index patterns to determine the cardiac condition of the animal being diagnosed.

2. The method according to claim 1, wherein the acquiring step is perform by acquiring the electrocardiographic signals as a function of time from a surface of the body of the animal being diagnosed through a plurality of surface electrodes over a multi-cycle test period.

3. The method according to claim 2. wherein the acquiring step is performed by processing the electrocardiographic signals into an analog signal that is indicative of the cardiac activity of the ventricle, and into another analog signal that is indicative of the cardiac activity of the heart.

4. The method according to claim 2. wherein the mathematically determining step is performed by converting the electrocardiographic time-dependent signals into respective power-spectrum characteristics wherein power is a function of frequency.

5. The method according to claim 4. wherein the establishing step is performed by setting in advance pre-set parameters, derived from the characteristic of wave-form of power spectrum; and wherein the recognizing step is performed by comparing each power spectrum characteristic against the pre-set parameters, and by determining the state of each index for each power spectrum characteristic in response to the comparing step.

6. The method according to claim 2. wherein the mathematically determining step is performed by converting the electrocardiographic time-dependent signals into a phase angle characteristic wherein relative phase shift between the electrocardiographic signals is a function of frequency.

7. The method according to claim 6. wherein the establishing step is performed by setting in advance pre-set parameters, derived from the characteristic of wave-form of the phase shift; and wherein the recognizing step is performed by comparing the phase angle characteristic against the pre-set parameters, and by determining the state of each index for the phase angle characteristic in response to the comparing step.

8. The method according to claim 2. wherein the mathematically determining step is performed by converting the electrocardiographic time-dependent signals into an impulse response characteristic wherein amplitude is a function of impulse time.

9. The method according to claim 8. wherein the establishing step is performed by setting in advance pre-set parameters, derived from the characteristic of wave-form of the impulse response; and wherein the recognizing step is performed by comparing the impulse response characteristic against the pre-set parameters, and by determining the state of each index for the impulse response characteristic in response to the comparing step.

10. The method according to claim 2. wherein the mathematically determining step is performed by converting the electrocardiographic time-dependent signals into a coherence characteristic wherein coherence between the electrocardiographic signals is a function of frequency.

11. The method according to claim 10. wherein the establishing step is performed by setting in advance pre-set parameters, derived from the characteristic of wave-form of the coherence; and wherein the recognizing step is performed by comparing the coherence characteristic against the pre-set parameters, and by determining the state of each index for the coherence characteristic in response to the comparing step.

12. The method according to claim 2. wherein the mathematically determining step is performed by converting the electrocardiographic time-dependent signals into an amplitude histogram characteristic wherein occurrence frequency is a function of amplitude.

13. The method according to claim 12. wherein the establishing step is performed by setting in advance pre-set parameters, including amplitude thresholds, interval between peaks and numbers of amplitudes; and wherein the recognizing step is performed by comparing each amplitude histogram characteristic against the pre-set parameters, and by determining the state of each index for each amplitude histogram characteristic in response to the comparing step.

14. The method according to claim 2. wherein the mathematically determining step is performed by converting the electrocardiographic time-dependent signals into a cross correlation characteristic between the electrocardiographic signals wherein cross correlation is a function of time shift between the electrocardiographic signals..

15. The method according to claim 14. Wherein the establishing step is performed by setting in advance pre-set parameters, derived from the characteristic of wave-form of the cross correlation; and wherein the recognizing step is performed by comparing the cross correlation characteristic against the pre-set parameters, and by determining the state of each index for the cross correlation characteristic in response to the comparing step.

16. The method according to claim 1. Wherein the generating step is performed by generating the integrated pattern from all of said functions.

17. An arrangement for diagnosing a cardiac condition of an animal, comprising:
(a) means for acquiring electrical analog electrocardiographic signals from the animal;
(b) means for mathematically determining the function descriptive of the animal heart from the analog signals;
(c) means for establishing a set of indices for each function, each indicative of the cardiac condition of the animal;
(d) means for recognizing the state of each index for each function;
(e) means for generating an integrated pattern of the states of the indices from the function;
(f) means for storing a collection of index patterns, each containing a multitude of patterns of the states of indices for a multitude of animals whose cardiac condition is known; and
(g) means for matching the generated integrated pattern against the stored collection of index patterns to determine the cardiac condition of the animal being diagnosed.
